(19) United States
(12) Patent Application Publication
(10) Pub. No.: US 2021/0299227 A1
(43) Pub. Date: Sep. 30, 2021

(54) Growth-Promoting Supplement for Livestock

(71) Applicant: Janine Alyn Hunt, Luling, TX (US)

(72) Inventor: Janine Alyn Hunt, Luling, TX (US)

(21) Appl. No.: 17/001,497

(22) Filed: Aug. 24, 2020

Related U.S. Application Data
(60) Provisional application No. 63/003,033, filed on Mar. 31, 2020.

(51) Int. Cl. A61K 38/39; A61K 31/4188; A61K 31/355; A61K 31/375; A61K 31/198; A61K 31/202; A61K 47/46; A61K 33/30; A23K 20/147; A23K 20/174; A23K 10/30; A23K 20/158; A23K 20/20; A23K 50/20

(52) U.S. Cl. CPC A61K 38/39; A61K 31/4188; A61K 31/355; A61K 31/375; A61K 31/198; A61K 31/202; A23K 50/20; A61K 33/30; A23K 20/147; A23K 20/174; A23K 10/30; A23K 20/158; A23K 20/30; A61K 47/46

(57) ABSTRACT

A growth-promoting supplement for livestock is a meal additive for hair growth, tail growth, and nail growth. The growth-promoting supplement also promotes hair, tail, and nail strength. The growth-promoting supplement includes a quantity of gelatin, a quantity of Biotin, a quantity of Lysine, a quantity of Vitamin E, and a quantity of Methionine. The composition is heterogeneously mixed into a powdered mixture that may be mixed into a meal for livestock. The composition further includes a quantity of alfalfa powder. Alternatively, the composition further includes a quantity of current-health-based additives. The current-health-based additives includes a quantity of Vitamin C, a quantity of Zinc, a quantity of Omega-3 fatty acids, and combinations thereof. Both the quantity of alfalfa powder and the quantity of current-health-based additives are heterogeneously mixed into the powdered mixture according the specific dietary needs and health condition of the livestock.

The current application claims a priority to the U.S. provisional patent application Ser. No. 63/003,033 filed on Mar. 31, 2020.

### FIELD OF THE INVENTION

The present invention generally relates to animal health supplements. More specifically, the present invention is a growth-promoting supplement for livestock.

### BACKGROUND OF THE INVENTION

The overall health of livestock is essential for production and performance. The overall health of a horse is seen in the quality of the mane, the tail, and the hooves of a horse. Any slight deficiencies for a horse are especially reflected in the condition of the mane, the tail, and the hooves. The damage to specifically the hooves of a horse as a result, may lead to more severe injuries and health problems.

An objective of the present invention is to provide a meal supplement that promotes growth and increases strength for the mane, tail, and hooves of livestock, specifically horses. Moreover, the present invention is a growth-promoting supplement for equine health supplement. The present invention can be provided in different forms for the consumption or administration of the supplement to the horse, preferably a powdered mixture. The horse supplement includes gelatin, Biotin, Lysine, Vitamin E, and Methionine. The present invention also preferably includes alfalfa powder. Depending on further dietary needs of the horse, the present invention may further include other moderate sources of vitamin C, Zinc, and omega 3 fatty acids. The present invention may be administered in different concentrations with different mass percent compositions specially made for different breeds, ages, health conditions, etc. In further embodiments, the present invention can be modified for different animals, such as cattle or other farm animals.

### BRIEF DESCRIPTION OF THE DRAWINGS

FIG. 1 is a block diagram of the present invention.

FIG. 2 is a block diagram of the preferred embodiment present invention with a quantity of alfalfa powder.

FIG. 3 is a block diagram of an alternate embodiment of the present invention with a quantity of current-health-based additives.

FIG. 4 is a block diagram illustrating the proportions of the present invention.

FIG. 5 is a block diagram illustrating the proportions of the preferred embodiment of the present invention.

### DETAIL DESCRIPTIONS OF THE INVENTION

All illustrations of the drawings are for the purpose of describing selected versions of the present invention and are not intended to limit the scope of the present invention.

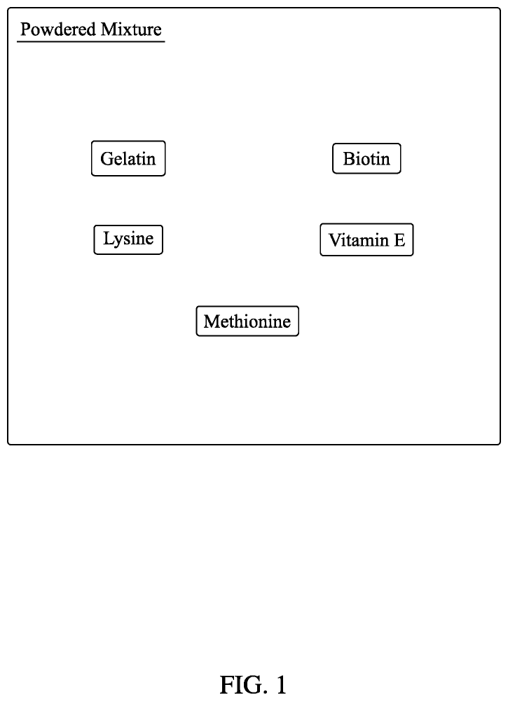

The present invention is a growth-promoting supplement for livestock. More specifically, the present invention is preferably a supplement for horses. The present invention may also be utilized as a supplement for a variety of other livestock such as cattle, sheep, goats, chickens, and so on. The present invention enhances the natural growth of a mane, a tail, and hooves of a livestock. The present invention repairs brittle manes and tails, patchy coats, and damaged hooves. Moreover, the present invention can serve as a preventative treatment as well as a recovery remedy for the mane, tail, and hooves of a livestock. As seen in FIG. 1, the composition of the growth-promoting supplement for livestock may comprise a quantity of gelatin, a quantity of Biotin, a quantity of Lysine, a quantity of Vitamin E, and a quantity of Methionine. The quantity of gelatin provides amino acids, which are essential for collagen production. Furthermore, the quantity of gelatin serves as a building block for cartilage production and provides essential amino acids. In the preferred embodiment of the present invention, the quantity of gelatin is pork gelatin so that the livestock consuming the present invention does not ingest any biproduct of itself. The quantity of Biotin is a vitamin that aids many functions of the body for a livestock. The quantity of Lysine and the quantity of Methionine are essential amino acids that are essential for sufficient protein production in the body of a livestock. The quantity of Vitamin E is a fat-soluble vitamin that provides antioxidant properties for a livestock. Thus, the quantity of gelatin, the quantity of Biotin, the quantity of Lysine, the quantity of Vitamin E, and the quantity of Methionine are heterogeneously mixed into a powdered mixture. The powdered mixture promotes and aid in protein production that is essential for general maintenance of overall health and reparative functions of a livestock.

The powdered mixture promotes hoof growth with the quantity of gelatin, the quantity of Biotin, and the quantity of Lysine. The quantity of Biotin specifically aids in the repair of weak or cracked hooves, and therefore promote hoof strength, along with the quantity of Lysine and the quantity of methionine. Furthermore, the quantity of gelatin and the quantity of Biotin also promote hair growth such as the hair of a mane or a tail. The quantity of Biotin specifically supplements coat growth as well. The powdered mixture serves to strengthen bones and ligaments with the quantity of Lysine as well. Moreover, the quantity of Lysine supplements any L-Lysine deficiencies of the diet of a horse. The quantity of Vitamin E promotes neuromuscular health. The powdered mixture also maintains the health of coat, skin, muscle, and hoof health with the quantity of Methionine.

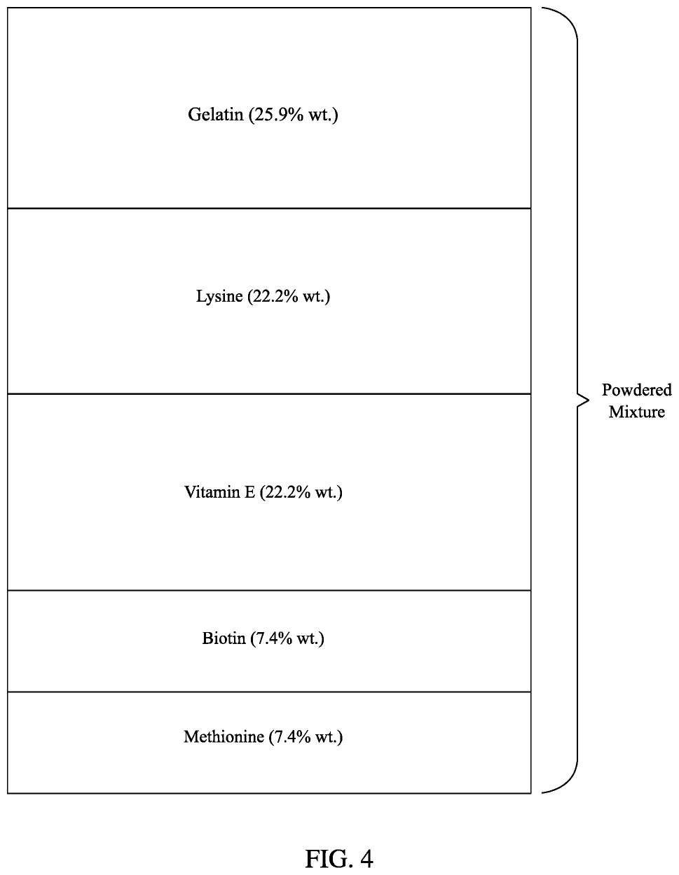

As can be seen in FIG. 4, in order to properly proportion the growth-promoting supplement that increases the daily average consumption of nutrition that promotes growth, the quantity of gelatin is approximately 25.9 percentage by weight (wt. %) of the powdered mixture. The quantity of Biotin is approximately 7.4 wt. % of the powdered mixture. The quantity of Lysine is approximately 22.2 wt. % of the powdered mixture. The quantity of Vitamin E is approximately 22.2 wt. % of the powdered mixture. The quantity of Methionine is approximately 7.4 wt. % of the powdered mixture. In reference to the aforementioned compositional proportions, the term “approximately” preferably means within an error range of up to ±0.05 wt. %. It is understood that slight adjustments with the amount of each ingredient are meant to accommodate the type of livestock, the current health status of the livestock, and the desired growth of the livestock.

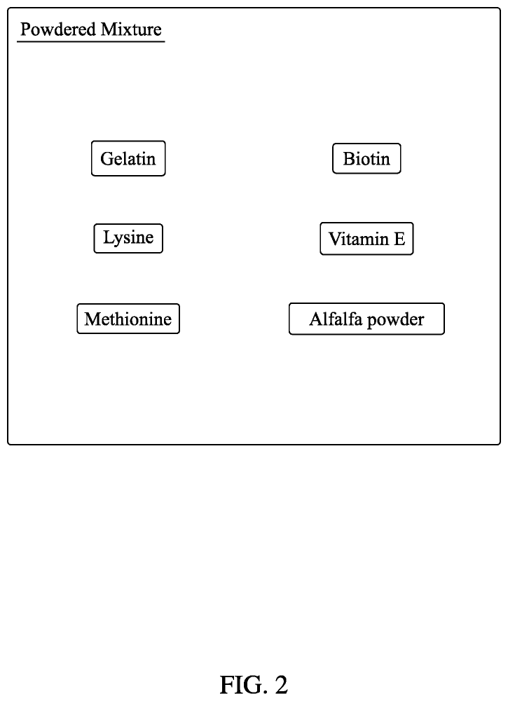

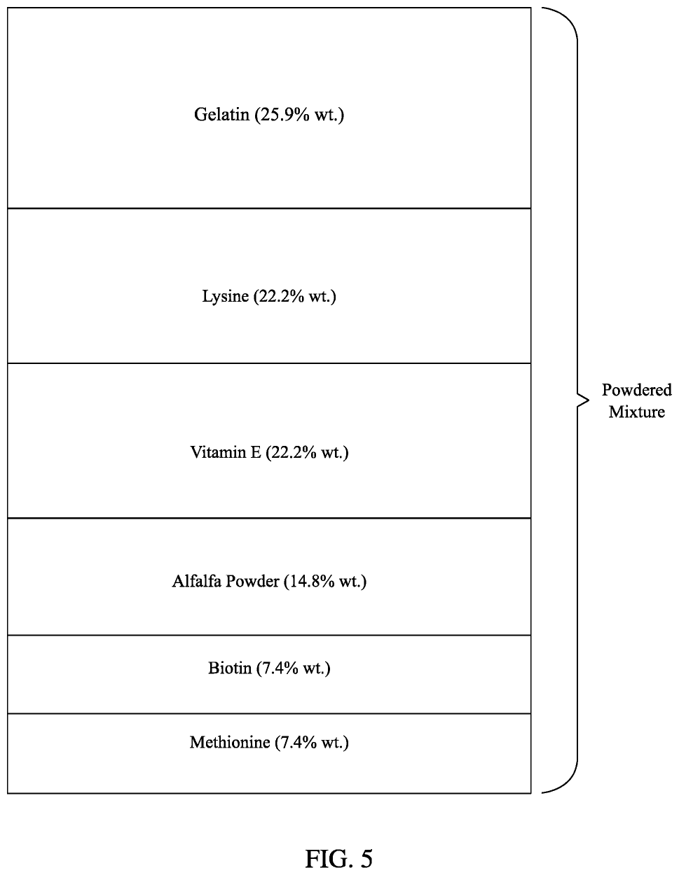

The preferred composition of the growth-promoting supplement may further comprise a quantity of alfalfa powder, seen in FIG. 2. The quantity of alfalfa powder is heterogeneously mixed into a powdered mixture and is approximately 14.8 wt. % of the powdered mixture, as seen in FIG. 5. The powdered mix further supplements the meal and current diet of a livestock as the quantity of alfalfa powder is a source of both protein and calcium.

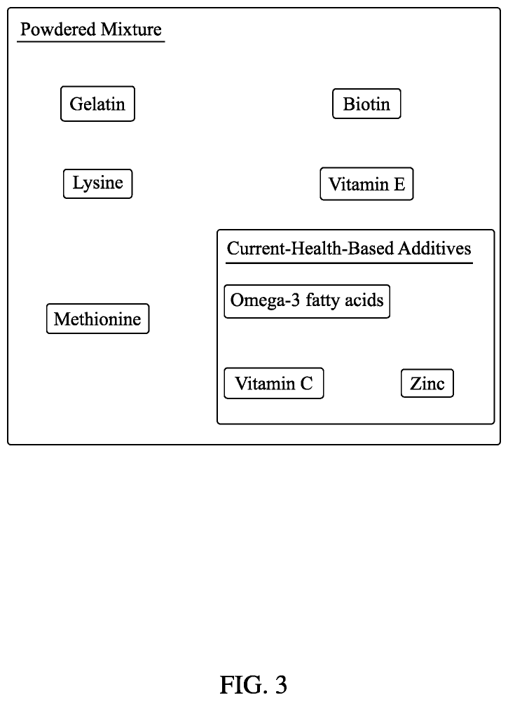

An alternate embodiment of the present invention comprises a quantity of current-health-based additives depending on the specific dietary needs of a livestock, seen in FIG. 3. The quantity of current-health-based additives is heterogeneously mixed into the powdered mixture. More specifically, the quantity of current-health-based additives includes at least one compositional constituent selected from a group consisting of a quantity of Vitamin C, a quantity of Zinc, a quantity of Omega-3 fatty acids, and combinations thereof. The quantity of Vitamin C contributes to the current supply of Vitamin C of a livestock which is used to create protein. The protein is then utilized for hair growth. The quantity of Zinc also serves to promote hair growth as well as hair repair. Moreover, the quantity of Zinc speeds the recovery of improper protein synthesis that is essential for overall hair growth. A livestock with a zinc deficiency benefits from the quantity of current-health-based additives because of the quantity of Zinc. The quantity of Omega-3 fatty acids provides anti-inflammatory properties for the quantity of current-health-based additives. More specifically, the quantity of Omega-3 fatty acids is a healthy fat that enriches the skin of a livestock. Both the quantity of Zinc and the quantity of Omega-3 fatty acids also supplement both the growth and strength for a hoof of a livestock.

The method of manufacturing includes the thorough mixing of the quantity of gelatin, the quantity of Biotin, the quantity of Lysine, the quantity of Vitamin E, and the quantity of Methionine. The powered mixture may be thoroughly mixed with a mixing mechanism and divided according to the mass and health condition of livestock. The method of use includes the administration of the powdered mixture to the meal of the livestock. The powdered mixture may be consumed daily depending on the existing diet and current health condition of livestock. Any excess powdered mixture may be packaged and stored for later consumption. It is understood that in further embodiments of the present invention, the powdered mixture may be further processed into various forms in order to facilitate the consumption of the present invention by livestock. The various forms may include, but are not limited to, tablets, pills, liquid supplement, and liquid injection.

Although the invention has been explained in relation to its preferred embodiment, it is to be understood that many other possible modifications and variations can be made without departing from the spirit and scope of the invention as hereinafter claimed.

What is claimed is:

1. A growth-promoting supplement for livestock comprises:
a quantity of gelatin;
a quantity of Biotin;
a quantity of Lysine;
a quantity of Vitamin E;
a quantity of Methionine; and,
the quantity of gelatin, the quantity of Biotin, the quantity of Lysine, the quantity of Vitamin E, and the quantity of Methionine being heterogeneously mixed into a powdered mixture.

2. The growth-promoting supplement for livestock as claimed in claim 1 comprises:
the quantity of gelatin being approximately 25.9 percentage by weight (wt. %) of the powdered mixture.

3. The growth-promoting supplement for livestock as claimed in claim 1 comprises:
the quantity of Biotin being approximately 7.4 wt. % of the powdered mixture.

4. The growth-promoting supplement for livestock as claimed in claim 1 comprises:
the quantity of Lysine being approximately 22.2 wt. % of the powdered mixture.

5. The growth-promoting supplement for livestock as claimed in claim 1 comprises:
the quantity of Vitamin E being approximately 22.2 wt. % of the powdered mixture.

6. The growth-promoting supplement for livestock as claimed in claim 1 comprises:
the quantity of Methionine being approximately 7.4 wt. % of the powdered mixture.

7. The growth-promoting supplement for livestock as claimed in claim 1, wherein the quantity of gelatin is pork gelatin.

8. The growth-promoting supplement for livestock as claimed in claim 1 comprises:
a quantity of alfalfa powder; and,
the quantity of alfalfa powder being heterogeneously mixed into a powdered mixture.

9. The growth-promoting supplement for livestock as claimed in claim 8 comprises:
the quantity of alfalfa powder being approximately 14.8 wt. % of the powdered mixture.

10. The growth-promoting supplement for livestock as claimed in claim 1 comprises:
a quantity of current-health-based additives; and,
the quantity of current-health-based additives being heterogeneously mixed into the powdered mixture.

11. The growth-promoting supplement for livestock as claimed in claim 10, wherein the quantity of current-health-based additives includes at least one compositional constituent selected from a group consisting of: a quantity of Vitamin C, a quantity of Zinc, a quantity of Omega-3 fatty acids, and combinations thereof.

12. A growth-promoting supplement for livestock comprises:
a quantity of gelatin;
a quantity of Biotin;
a quantity of Lysine;
a quantity of Vitamin E;
a quantity of Methionine;
a quantity of alfalfa powder;
a quantity of current-health-based additives; and,
the quantity of gelatin, the quantity of Biotin, the quantity of Lysine, the quantity of Vitamin E, the quantity of Methionine, the quantity of alfalfa powder, and the quantity of current-health-based additives being heterogeneously mixed into a powdered mixture.

13. The growth-promoting supplement for livestock as claimed in claim 12 comprises:
the quantity of gelatin being approximately 25.9 percentage by weight (wt. %) of the powdered mixture.

14. The growth-promoting supplement for livestock as claimed in claim 12 comprises:
the quantity of Biotin being approximately 7.4 wt. % of the powdered mixture.

15. The growth-promoting supplement for livestock as claimed in claim 12 comprises:
the quantity of Lysine being approximately 22.2 wt. % of the powdered mixture.

16. The growth-promoting supplement for livestock as claimed in claim 12 comprises:
the quantity of Vitamin E being approximately 22.2 wt. % of the powdered mixture.

17. The growth-promoting supplement for livestock as claimed in claim 12 comprises:
the quantity of Methionine being approximately 7.4 wt. % of the powdered mixture.

18. The growth-promoting supplement for livestock as claimed in claim 12, wherein the quantity of gelatin is pork gelatin.

19. The growth-promoting supplement for livestock as claimed in claim 12 comprises:
the quantity of alfalfa powder being approximately 14.8 wt. % of the powdered mixture.

20. The growth-promoting supplement for livestock as claimed in claim 12, wherein the quantity of current-health-based additives includes at least one compositional constituent selected from a group consisting of: a quantity of Vitamin C, a quantity of Zinc, a quantity of Omega-3 fatty acids, and combinations thereof.
